(19) United States
(12) Patent Application Publication
(10) Pub. No.: US 2015/0038677 A1
(43) Pub. Date: Feb. 5, 2015

(54) PROCESS FOR THE SYNTHESIS OF TELAPREVIR, OR PHARMACEUTICALLY ACCEPTABLE SALTS OR SOLVATES AS WELL AS INTERMEDIATE PRODUCTS THEREOF

(71) Applicant: SANDOZ AG, Basel (CH)

(72) Inventors: Wolfgang Felzmann, Kundl (AT); Stefanie Brunner, Kundl (AT); Thorsten Wilhelm, Kundl (AT)

(21) Appl. No.: 14/384,268

(22) PCT Filed: Mar. 15, 2013
(86) PCT No.: PCT/EP2013/055397

§ 371 Date: Sep. 10, 2014

(30) Foreign Application Priority Data
Mar. 16, 2012 (EP) 12159923.7

(51) Int. Cl. C07K 5/117; C07K 5/08

(52) U.S. Cl. CPC C07K 5/1024; C07K 5/08; C07B 2200/13
USPC 530/330

(57) ABSTRACT

The invention relates to a process for the preparation of telaprevir, or a pharmaceutically acceptable salt or solvate thereof, wherein the process requires a smaller number of process steps and/or does not require the use of toxic and instable compounds compared to the known processes. Another embodiment refers to telaprevir, or a pharmaceutically acceptable salt or solvate thereof as well as to intermediate products for preparation of the same, wherein the afore-mentioned products are obtained by the process described herein.

The invention relates to a process for the preparation of telaprevir or a pharmaceutically acceptable salt or solvate thereof, wherein the process requires a smaller number of process steps and/or does not require the use of toxic and instable compounds compared to the known processes. Another embodiment refers to telaprevir or a pharmaceutically acceptable salt or solvate thereof as well as to an intermediate product for preparation of the same, wherein the afore-mentioned products are obtained by the process described herein.

### BACKGROUND PRIOR ART

Telaprevir is a protease inhibitor that can be used as antiviral drug. By way of example, telaprevir inhibits the hepatitis C virus NS3-4A serine protease.

Although some processes for the synthesis of telaprevir or its pharmaceutical acceptable salts are available, it is an object of the present invention to provide an alternative process, in particular an enhanced process that overcomes at least one of the problems of the prior art processes.

Y. Yip et al. Bioorg. Med. Chem. Lett., 2004, 14, 5007 discloses the preparation of a 1:1 mixture of isomers defined by Formula 5a (see Scheme 1) which isomers appear to have a stereochemical configuration other than that of telaprevir.

WO 2007/022459 A2 discloses a process for preparing telaprevir, wherein in a first coupling step, a bicyclic pyrrolidine derivative is reacted with a protected amino acid, followed by a step-wise extension of the chain of the amino acid to provide a tripeptide as shown in Formula 2. Subsequently, a β-amino acid is added to the carbon chain-end opposite to said previously built chain. Finally, telaprevir is obtained in an oxidation step.

Turner et al. (Chemical Communications 2010, 46(42), 7918) discloses a process for the preparation of telaprevir by applying an Ugi reaction type process which reacts a compound of Formula 2

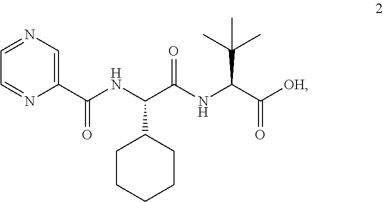 [SMILES: CC(C)(C)[C@H](NC(=O)[C@@H](NC(=O)c1cnccn1)C1CCCCC1)C(=O)O]

a chiral imine, namely (3aS,6aR)-1,3a,4,5,6,6a-hexahydrocyclopenta[c]pyrrole, which is obtained by enzyme technology and is thus difficult to prepare and is instabile and a relatively unstable isonitrile derivative of formula 4.

### SUMMARY OF THE INVENTION

The known processes for the preparation of telaprevir are based on long linear sequences or require the use of labile, highly reactive agents and specific enzymes. The process described herein may for example allow to avoid the use of said labile, highly reactive reactants and specific enzymes.

It was surprisingly found within the context of the present invention that telaprevir may be prepared in a smaller number of process steps in a convergent manner by using stabile precursors (see an example process in FIG. 1). The present invention may also contribute to preserving the desired stereochemical configuration during the process of preparing telaprevir.

In particular, it has been found that the desired stereochemical configuration may be preserved during the process of peptide bond formation in the compound according to Formula 5 when using the coupling agents described herein, in particular when using 2,4,6-tripropyl-1,3,5,2,4,6-trioxatriphosphorinane-2,4,6-trioxide (T3P) or related compounds in dichloromethane.

It is also possible to use a combination of a diimide coupling reagent, including but not being limited to dicyclohexylcarbodiimide (DCC), diispropylcarbodiimide (DIC) and 1-ethyl-3-(3-dimethylaminopropyl)carbodiimide hydrochloride (EDC), with 1-hydroxy-benzotriazole (HOBt) or 1-hydroxy-7-aza-benzotriazole (HOAt) or related reagents for preparing telaprevir.

It has been found that the coupling agents are particularly effective when used in the presence of a lewis acid such as a copper salt. It was also unexpectedly found that the choice of solvent for carrying out the coupling reaction may further enhance the preservation of the stereochemical configuration during peptide bond formation in the compound according to Formula 5.

Furthermore, the expensive compound according to Formula 3

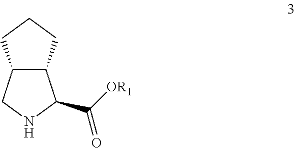

is used at a later stage of the process compared to the process of WO 2007/022459 A2, namely for coupling to the compound according to Formula 2 which already represents a dipeptide. Considering the yields of the single process steps, a smaller amount of the compound according to Formula 3 is required according to the invention, and, thus, the process may be less costly. Compared to the above method of Turner et al., it is not required to use a toxic and instable isonitrile compound.

It has also been found that the process for preparing telaprevir may provide an advantage since fewer impurities such as epimeric forms and other byproducts may be formed.

Thus, one embodiment provides a process for the preparation of telaprevir according to Formula 1

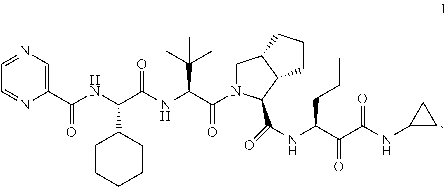 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

or a pharmaceutically acceptable salt or solvate thereof, comprising the steps of:

i) providing a compound according to Formula 2

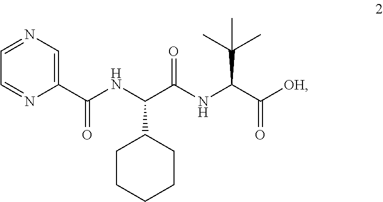 [SMILES: CC(C)(C)[C@H](NC(=O)[C@@H](NC(=O)c1cnccn1)C1CCCCC1)C(=O)O]

ii) bringing the compound according to Formula 2 into contact with a compound according to Formula 3

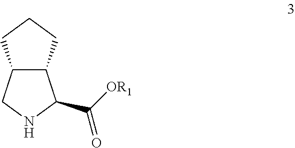

wherein R₁ is a protection group,

in the presence of one or more coupling agents, thereby obtaining a compound according to Formula 5

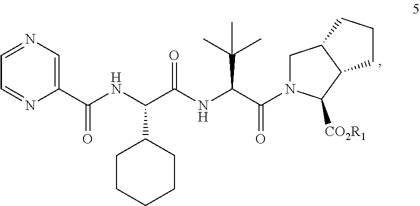

iii) deprotecting the compound according to Formula 5 by removing the R₁ protection group in order to obtain an acid according to Formula 7

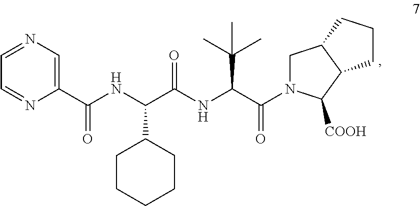 [SMILES: CC(C)(C)[C@H](NC(=O)[C@@H](NC(=O)c1cnccn1)C1CCCCC1)C(=O)N1C[C@@H]2CCC[C@@H]2[C@H]1C(=O)O]

iv) bringing the acid according to Formula 7 into contact with a compound according to Formula 4

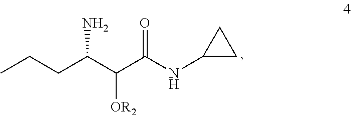

wherein R₂ is H or a suitable protecting group,

in the presence of one or more coupling agents,

thereby obtaining a compound according to Formula 6

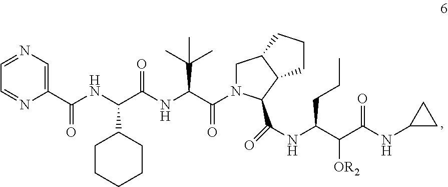

wherein R₂ is H, or optionally, a suitable protecting group

followed by removal of the optionally present protecting group R₂,

thereby obtaining a compound according to Formula 6 wherein R₂ is H

v) oxidizing the compound according to Formula 6 wherein R₂ is H thereby obtaining telaprevir according to Formula 1, or a pharmaceutically acceptable salt or solvate thereof.

A further aspect is a process for the preparation of a compound according to Formula 5 comprising the steps of:

i) providing a compound according to Formula 2,

ii) bringing the compound according to Formula 2, into contact with a compound according to Formula 3, wherein R₁ is a protection group, in the presence of one or more coupling agents, thereby obtaining a compound according to Formula 5. The preferred embodiments of this process are described in respect to the preparation of telaprevir (see particularly steps (i) and (ii) of the preparation of telaprevir) above.

Another embodiment is a process for the preparation of a pharmaceutical composition or pharmaceutical dosage form comprising telaprevir according to Formula 1 or a pharmaceutically acceptable salt or solvate thereof, comprising the process steps as described herein and further comprising formulating the obtained telaprevir according to Formula 1, or a pharmaceutically acceptable salt or solvate thereof into a pharmaceutical composition or pharmaceutical dosage form.

A further embodiment is a compound according to Formula 5

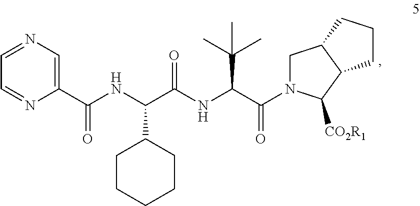

wherein R₁ is a protection group,

obtainable or obtained by carrying out steps (i) to (ii) of the process as described herein.

A further embodiment is a crystalline compound according to Formula 7

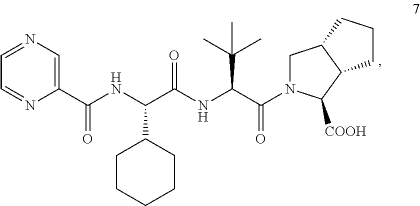

wherein R₁ is H,

obtainable or obtained by carrying out steps (i) to (iii) of the process as described herein.

A further embodiment is a compound according to Formula 6

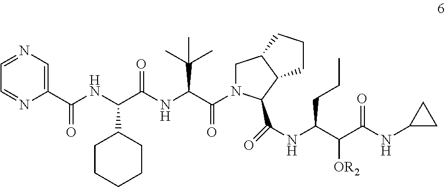

wherein R₂ is H

obtainable or obtained by carrying out steps (i) to (iv) of the process as described herein.

Another embodiment is telaprevir according to Formula 1, or a pharmaceutically acceptable salt or solvate thereof, obtainable or obtained by the process as described herein.

Yet another embodiment is telaprevir, or a pharmaceutically acceptable salt or solvate thereof having an epimeric impurity of less than 0.15% at the tert-leucine position in Formula 1.

### LIST OF FIGURES

FIG. 1: Shows a reaction scheme for the synthesis of telaprevir according to the invention.

FIG. 2: Arrow indicates the tert-leucine position in telaprevir according to Formula 1.

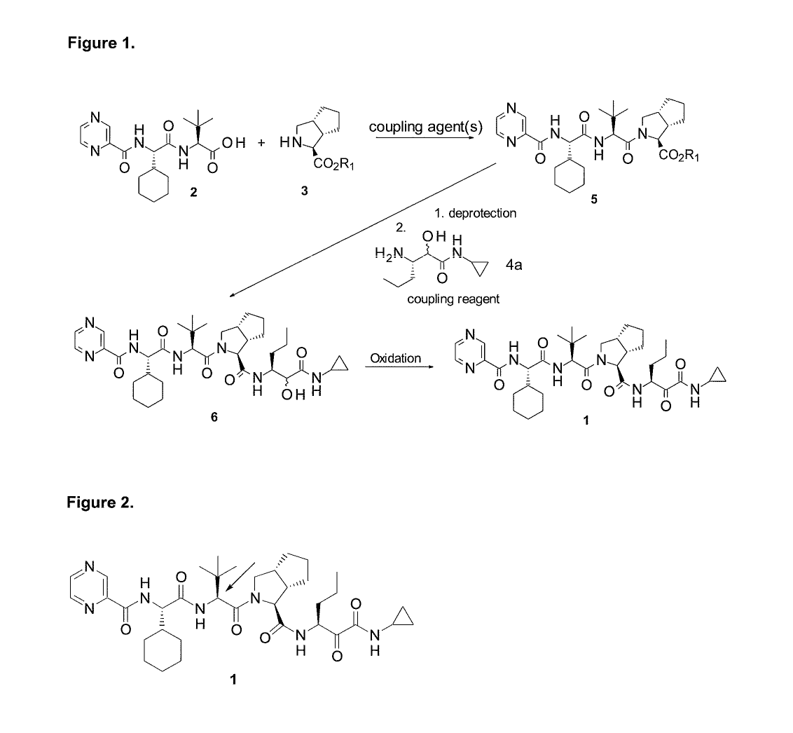

### DETAILED DESCRIPTION

The invention relates to a process for the preparation of telaprevir according to Formula 1

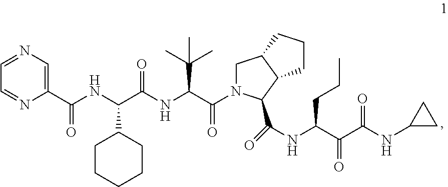 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

or a pharmaceutically acceptable salt or solvate thereof, wherein the process comprises the steps of (i) to (v). In the process described herein, preferably, telaprevir according to formula 1 is prepared via the compounds according to Formulas 2-7.

Pharmaceutically acceptable salts include, but are not limited to the group consisting of hydrochloride, hydrobromide, sulphates or phosphates as well as organic salts such as acetate, citrate, maleate, succinate, and lactate, benzoate. Pharmaceutically acceptable salts can be obtained according to standard methods, for example by addition of the respective acid to telaprevir as free base.

In step (i), a compound according to Formula 2

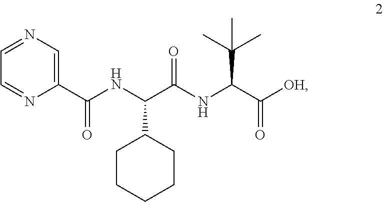 [SMILES: CC(C)(C)[C@H](NC(=O)[C@@H](NC(=O)c1cnccn1)C1CCCCC1)C(=O)O]

is provided.

Preferably, step (i) includes dissolving the compound according to Formula 2 in a solvent or mixture of solvents. However, it is also possible to add the compound according to Formula 2 in solid form to the compounds of step (ii). Suitable solvents can be chosen by a person skilled in the art according to common practice. Preferably, inert solvents are used. The term “inert solvent” refers to any solvents that do not react with the compounds of Formulas 1-7. Inert solvents suitable in this respect are commonly known. Additionally preferred, the solvent(s) used in step (i) and/or step (iv) is/are selected from the group consisting of ethylacetate, dichloromethane, N,N-dimethylacetamide, dimethyl sulfoxide (DMSO), N-methylpyrrolidone, acetonitrile, methyl tert-butyl ether, tetrahydrofuran, 2-methyltetrahydrofuran, toluene and N,N-dimethylformamide, preferably ethylacetate, N-methylpyrrolidone, N,N-dimethylacetamide, N,N-dimethylformamide, toluene, methyl tert-butyl ether, 2-methyltetrahydrofuran, or dichloromethane, more preferably toluene, N-methylpyrrolidone, N,N-dimethylacetamide, N,N-dimethylformamide, methyl tert-butyl ether, 2-methyltetrahydrofuran or dichloromethane, and most preferably N,N-dimethylacetamide, N,N-dimethylformamide, and dichloromethane. The afore-mentioned solvents or mixtures thereof may also be used in other steps of the process described herein, where applicable.

The compound according to Formula 2 can be prepared by applying standard peptide synthesis methods (see e.g. Turner et al., Chem. Commun., 2010, 46, 7918-7920; Y. Yip et al. Bioorg. Med. Chem. Lett., 2004, 14, 5007).

The compound according to Formula 2 preferably used in stereochemically pure form, based on synthesis from enantiomerically enriched amino-acid building blocks. Preferably, the compound of Formula 2 has a diastereomeric purity of at least 70%, preferably 80%, further preferred 90%, even further preferred 95% and most preferably more than 97% based on the total amount of all isomers of Formula 2.

Step (ii) comprises bringing the compound according to Formula 2 of step (i) into contact with a compound according to Formula 3

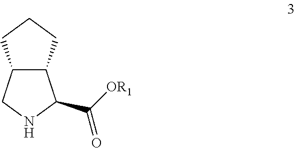

wherein R₁ is a protection group, in the presence of one or more coupling agents, preferably in the presence of a solvent, thereby obtaining a compound according to Formula 5

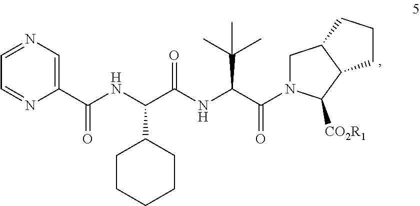

In the compounds according to Formula 3 and/or 5, R₁ can be chosen to form an ester protecting group, preferably R₁ is a saturated or unsaturated, substituted or unsubstituted, branched or linear, C₁₋₁₀, preferably C₁₋₆, hydrocarbon compound. Further preferred, R₁ is selected from the group consisting of tert-butyl (compounds 3a/5a as depicted in the experimental section), methyl, ethyl (compounds 3b/5b in the experimental section), propyl, iso-propyl, butyl, isobutyl, benzyl, vinyl, 1-propenyl and allyl.

Examples of acid protecting groups for the purpose of the invention are for example described in T. W. Greene & P. G. M Wutz, “Protective Groups in Organic Synthesis,” 3rd Edition, John Wiley & Sons, Inc. (1999). The compound of Formula 3 can be prepared by using the process described in WO 2007/022459 A2 (paragraphs [00140]-[00145]), or alternative methods known to those skilled in the art.

The compound according to Formula 3 may preferably be used in stereochemically pure form. Preferably, the compound of Formula 3 has a stereochemical purity of at least 70%, preferably of at least 80%, further preferred of at least 90%, even further preferred of at least 95% and most preferably more than 97% based on the total amount of all isomers of Formula 3. The stereochemical purity/enantiomeric purity can for example be determined by appropriate nuclear magnetic resonance (NMR) experiments as known in the art or by chiral high performance liquid chromatography (HPLC) as known in the art, as described above.

The step of bringing the compound according to Formula 2 into contact with a compound according to Formula 3 can for example be carried out by dissolving said compounds either separately or as a mixture of compounds or by dissolving one of the compounds and adding to this solution the respective other compound. The coupling agents can then be added. However, the order of combining the compounds can be altered.

The amount of the compound according to Formula 3 as well as the amount(s) of coupling agent(s) are calculated using specific molar ratios relative to the total amount of the compound according to Formula 2 and its stereoisomers. Furthermore, the amount of the compound according to Formula 3 is given as the amount of the compound according to Formula 3 and all stereoisomers thereof (depending on the purity of the compound according to Formula 3, further stereoisomers may be present and the weight of all stereoisomers is taken as a whole). This means that regarding the amounts of compounds used in the process and defined herein, the total amounts of all stereoisomers of the respective compound are taken as basis. The total amount of the compound according to Formula 3 and/or its stereoisomers in step (ii) is preferably from 0.8 to 3 equivalents, preferably from 0.9 to 2.0 equivalents, preferably from 1.0 to 1.6 equivalents, based on the total amount of the compound according to Formula 2 and its stereoisomers.

The amount of coupling agent(s) in step (ii) and/or step (iv) is from 0.8 to 6 equivalents, preferably from 0.9 to 4 equivalents, further preferred from 1 to 2 equivalents, based on the total amount of the compound according to Formula 2 and its stereoisomers. If more than one coupling agent is used, the different types of coupling agents can be used in the same or different amounts. Preferably, they are all used in amount of more than 1 equivalent based on the amount of the compound according to Formula 2 and its stereoisomers. Further preferred, each coupling agent is used in an amount of 1 to 2 equivalents based on the total amount of the compound according to Formula 2 and its stereoisomers.

If the compound according to Formula 3 is not used in the form of a free base, or if otherwise desirable, step (ii) can be carried out in the presence of an organic base, such as tertiary amine bases like diisopropylethylamine, N-methylmorpholine, and triethylamine, or an inorganic base such as potassium carbonate, sodium carbonate or sodium bicarbonate. Suitable amounts of base are for example 1-6 equivalents based on the total amount of the compound according to Formula 3 and its stereoisomers.

A suitable reaction temperature for step (ii) can be chosen by a person skilled in the art. For example, the step of combining the coupling agent(s) with the other compound can be carried out at 0° C. to room temperature (for example for a time of 1 minute to 1 hour) and the reaction can then be completed at 0° C. to 50° C. (for example for a time of 1 hour to 30 hours). Room temperature is defined herein as a temperature range of 20-25° C.

Suitable amount(s) of solvent(s) that is/are used in step (ii) can be chosen by a person skilled in the art. The use of lower amounts of solvents leading to higher concentrations may provide for a faster reaction rate.

The coupling agent(s) in steps (ii) and/or (iv) represent acid activation agents and allow for the formation of peptide bonds between the compounds according to Formula 2 and 3 and the compounds according to Formula 7 and 4, respectively.

A preferred coupling agent used in step (ii) is a substituted 1,3,5,2,4,6-trioxatriphosphorinane-2,4,6-trioxide, preferably a compound according to Formula 8

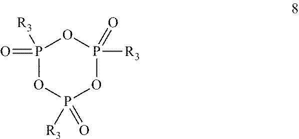

wherein R₃ is a saturated or unsaturated, cyclic, branched or linear, substituted or unsubstituted C₁₋₁₀ hydrocarbon compound, preferably, R₃ is n-propyl or phenyl

Thus, preferred coupling agents are 2,4,6-tripropyl-1,3,5,2,4,6-trioxatriphosphorinane-2,4,6-trioxide (T3P) and 2,4,6-triphenyl-1,3,5,2,4,6-trioxatriphosphorinane-2,4,6-trioxide. As shown in the experimental part, the use of T3P in combination with e.g. dichloromethane as solvent may provide for very good preservation of diasteromeric purity during formation of the compound according to Formula 5. If a compound according to Formula 8 is used as coupling agent, it is preferred to use only one type of coupling agent.

Also preferred coupling agents are carbodiimides. However, it is also possible to use other coupling agents known in the art such as uronium coupling agents. For an overview of possible coupling reagents, refer Han, S.-Y.; Kim, Y.-A. Tetrahedron 2004, 60, 2447-2467.

Carbodiimides are known in the art. For example, 1-ethyl-3-(3-dimethylaminopropyl)carbodiimide hydrochloride (EDC), N,N′-dicyclohexylcarbodiimide (DCC) or N,N′-diisopropylcarbodiimide (DIC) can be used. If the acid activation agent in step (ii) is a carbodiimide, it is preferred to use a second activation agent such as 1-hydroxy-benzotriazole (HOBt) or 1-hydroxy-7-aza-benzotriazole (HOAt) that reduces the reactivity of the carbodiimide by formation of an activated species which is less active than the species formed with the carbodiimide. Preferred carbodiimides are selected from the group consisting of N,N′-diisopropylcarbodiimide (DIC), N,N′-dicyclohexylcarbodiimide (DCC) and 1-ethyl-3-(3-dimethylaminopropyl)carbodiimide hydrochloride (EDC), preferably, N,N′-diisopropylcarbodiimide (DIC) is used. Furthermore, HOBt or HOAt can be used in solid-supported form.

Additionally preferred, step (ii) and/or (iv), preferably step (ii), is carried out in the presence of a lewis acid such as for example a copper salt, preferably CuCl₂. It is particularly preferred to use the aforementioned lewis acid if the coupling agent is a carbodiimide, in particular, when the carbodiimide is DIC. In one embodiment, a carbodiimide compound, preferably DIC, is used in combination with a lewis acid, preferably CuCl₂, in the absence of a triazole-based coupling reagent. The lewis acid can be used in an amount of from 1 to 6 equivalents, preferably of from 1.5 to 2.5 equivalents, or from 1 to 2 equivalents, based on the total amount of the compound according to Formula 2 and its stereoisomers, or based on the total amount of the compound according to Formula 7 and its stereoisomers (depending on whether it is used in step (ii) or (iv)). It is even possible to use smaller amounts of CuCl₂, such as 0.55 to 3 equivalents, preferably 0.55 to 1.5 equivalents, or 0.55 to 1 equivalents. It has unexpectedly been found that the use of a lewis acid, in particular CuCl₂, may contribute to preserving the diastereomeric purity during the peptide bond formation leading to the compound according to Formula 5.

Generally, step (ii) can include the isolation of the compound according to Formula 5. Suitable methods for isolating said compound are known in the art and comprise for example the washing of the organic layer with an aqueous salt solution (e.g. brine), separation of the organic layer, drying of said organic layer and removal of the organic solvent in vacuo. Dependent on the specific conditions used for step (ii) and/or (iv), the work-up may further include acid and/or base washes. Furthermore, the compound according to Formula 5 can purified by using flash chromatography prior to step (iii) as known in the art. However, it is more preferred to continue directly with step (iii) without isolation of said intermediate compound.

Additionally preferred, process step (ii) provides the compound according to Formula 5 with an epimeric impurity at the tert-leucine position of less than 20%. Even more preferred is less than 10% and even more preferred is less than 2% of said epimeric impurity.

In step (iii) the compound according to Formula 5 is deprotected by removing the R₁ protection group, preferably in the presence of a solvent, in order to obtain an acid according to Formula 7,

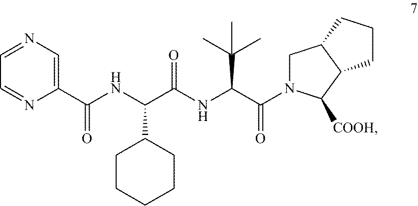 [SMILES: CC(C)(C)[C@H](NC(=O)[C@@H](NC(=O)c1cnccn1)C1CCCCC1)C(=O)N1C[C@@H]2CCC[C@@H]2[C@H]1C(=O)O]

The deprotecting agent and conditions for carrying out the deprotection reaction can be chosen according to common knowledge depending on the protecting group that is used (see e.g. Greene's Protective Groups in Organic Synthesis, Peter G. M. Wuts, Theodora W. Greene, 2007 John Wiley & Sons, Inc., Hoboken, N.J., Fourth Edition). In particular, the deprotecting agent used in step (iii) is chosen depending on the ester protection group that is used, preferably an alkali hydroxide base is used as deprotecting agent, further preferred is NaOH, especially in combination with an ethylester protecting group. As a solvent various solvents can be used for this reaction, especially water-miscible solvents, among these acetonitrile, tetrahydrofuran, ethanol, methanol, isopropanol, propanol, dioxane are preferred, an especially preferred solvent for the deprotection step (iii) is tetrahydrofuran (THF)/H₂O or ethanol/H₂O.

Preferably, step (iii) includes the isolation of the compound according to Formula 7. Additionally preferred, the compound according to Formula 7 is crystallised after a suitable work-up and purification. A suitable work-up is known to someone skilled in the art and may include extraction of the product into the aqueous layer using an aqueous base, followed by re-acidification and extraction of the aqueous layer using a suitable solvent like ethyl acetate. (iv).

After drying of the organic layer, the compound according to Formula 7 can be isolated according to standard methods known to those skilled in the art; preferably, the compound according to Formula 7 is crystalized by addition of an anti-solvent. A useful anti-solvent for this process can be, without being limited to, hexane, heptane or toluene. The crystalline compound according to Formula 7 has high purity, preferably has an amount of epimeric impurity at the tert-leucine position of less than 1.0%, preferably less than 0.5%. The smallest amount of epimeric impurity may for example be 0.05%. The epimeric impurities can be determined by HPLC-MS or NMR.

In step (iv) the acid according to Formula 7 is brought into contact with a compound according to Formula 4

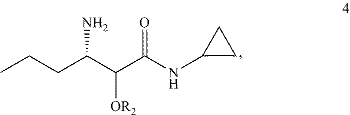

wherein R₂ is H or a protection group preferably in the presence of a solvent. Preferably, compound 4a is used:

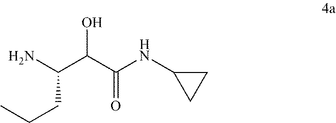

The acid according to Formula 7 is brought into contact with a compound according to Formula 4 in the presence of one or more coupling agents. Preferred coupling agents and amounts of coupling agents are described above. Furthermore, activation using chloroformate activating agents is also preferred. In step (iv), the amounts of compounds are calculated using specific molar ratios relative to the total amount of the compound according to Formula land its stereoisomers.

The compounds according to Formula 4 or 4a can for example be prepared by using a process which is analog to that described in Harbeson, S. L. et al. J. Med. Chem. 1994, 37, 2918-2929, or by using a process as described in Avolio, S. et al., Bioorg. Med. Chem. Lett. 2009, 19, 2295-2298 as well as WO 2007/022459 A2 (paragraphs [00148]-[00153]), or as described in WO2010/126881. The compound of Formula 4 can have a high isomeric purity with less than 10%, preferably less than 5%, of stereoisomers of the isomer of Formula 4. The isomeric purity can be determined by HPLC-MS.

The compound according to Formula 4 can be (2S,3S)-3-amino-N-cyclopropyl-2-hydroxyhexanamide or a derivative thereof where R₂ is a protecting group, (2R,3S)-3-amino-N-cyclopropyl-2-hydroxyhexanamide or a derivative thereof where R₂ is a protecting group, or a mixture thereof. Step (iv) provides a compound according to Formula 6

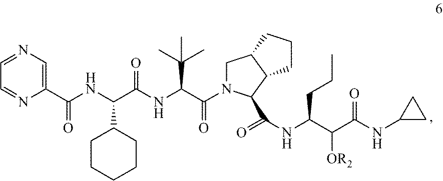

wherein R₂ is H or a protecting group, or provides a compound according to Formula 6a

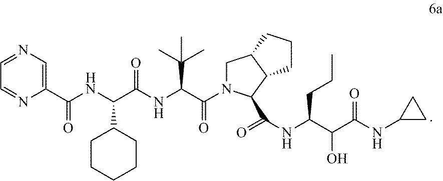 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(O)C(=O)NC1CC1]

If R₂ is a protecting group, it can be removed using methods known to someone skilled in the art, as described, for example in T. W. Greene & P. G. M Wutz, “Protective Groups in Organic Synthesis,” 3rd Edition, John Wiley & Sons, Inc. (1999), thereby obtaining a compound according to Formula 6a.

Possibly, step (iv) includes the isolation of the compound according to Formula 6 or 6a.

The total amount of the compound according to Formula 4 and/or its stereoisomers (depending on the purity of the compound according to Formula 4, further stereoisomers may be present) used in step (ii) is preferably from 0.8 to 3 equivalents, preferably from 0.9 to 1.5 equivalents, preferably from 0.9 to 1.2 equivalents, based on the total amount of the compound according to Formula 7 and its stereoisomers.

If the compound according to Formula 4 is not used in the form of a free base, or if otherwise desirable, step (iv) can be carried out in the presence of an organic base, such as tertiary amine bases like diisopropylethylamine, N-methylmorpholine, and triethylamine, or an inorganic base such as potassium carbonate, sodium carbonate or sodium bicarbonate. Suitable amounts of base are for example 1-6 equivalents based on the total amount of the compound according to Formula 4 and its stereoisomers.

A suitable reaction temperature for step (iv) can be chosen by a person skilled in the art. For example, the step of combining the compound according to Formula 4 with the other compounds can be carried out at 0° C. to room temperature (for example for a time of 1 minute to 1 hour) and the reaction can then be completed at 0° C. to 50° C. (for example for a time of 1 hour to 12 hours). Additionally preferred, after completion of the reaction, the reaction mixture is quenched by addition of water followed by acidification. Additionally preferred, the compound according to Formula 6/6a is then isolated by using the same or a similar method as described above.

The oxidizing agent in step (v) is known to someone skilled in the art, preferably it is selected from the group of hypervalent iodine oxidants, comprising but not being limited to the Dess-Martin periodinane (1,1,1-Tris(acetyloxy)-1,1-dihydro-1,2-benziodoxol-3-(1H)-one) or IBX (2-iodoxybenzoic acid), or sodium hypochlorite in the presence of 2,2,6,6-tetramethylpiperidinyloxy free radical (TEMPO). Preferably, the oxidizing agent is sodium hypochlorite in the presence of 2,2,6,6-tetramethylpiperidinyloxy free radical (TEMPO).

Suitable amounts of oxidizing agent(s) can be chosen by a person skilled in the art according to common practice. For example, the oxidizing agent can be used in an amount of 0.9-2 equivalents, preferably, from 0.9 to 1.2 equivalents, based on the total amount of the compound according to Formula 6/6a and its stereoisomers which total amount represents 1 equivalent. TEMPO can be used in catalytic amounts. Particular suitable is a combination of a catalytic amount of TEMPO with KBr, NaHCO₃, and NaOCl in dichloromethane.

Process steps (iii)-(v) can also be carried out as described in WO 2007/022459 A2.

In step (v), the compound according to Formula 6 is oxidized, preferably in the presence of a solvent, thereby obtaining telaprevir according to Formula 1, or a pharmaceutically acceptable salt or solvate thereof.

Step (v) can additionally comprise adding compounds such as acids to the reaction mixture to provide pharmaceutically acceptable salts of telaprevir.

Additionally preferred, step (v) comprises a further step of isolating telaprevir or a pharmaceutically acceptable salt or solvate thereof. Optionally, the obtained telaprevir or its pharmaceutically acceptable salt or solvate is precipitated and for example filtered off, washed with solvent and dried. Prior to isolating the product, a flash chromatography may be applied for purification. It is also preferred to isolate telaprevir, or a pharmaceutically acceptable salt or solvate thereof by crystallization.

By way of this invention, telaprevir obtained by this process contains a diastereomeric impurity at the tert-leucine position (cf. FIG. 2) of less than 1%. Even more preferred is less than 0.5% and even more preferred is less than 0.15% of said epimeric impurity. The smallest amount of epimeric impurity may for example be 0.05%.

Additionally preferred, the process as described herein provides telaprevir according to Formula 1, a pharmaceutically acceptable salt or solvate thereof in amorphous form, crystalline form, as a toluene solvate or as cocrystals.

A further aspect is a process for the preparation of a compound according to Formula 5, comprising the steps of:

i) providing a compound according to Formula 2,

ii) bringing the compound according to Formula 2 into contact with a compound according to Formula 3, wherein R₁ is a protection group, in the presence of one or more coupling agents, thereby obtaining a compound according to Formula 5. The preferred embodiments of this process are described in respect to the preparation of telaprevir (see particularly steps (i) and (ii) of the preparation of telaprevir) above.

Another aspect is the preparation of a pharmaceutical composition or pharmaceutical dosage form comprising telaprevir according to Formula 1, or a pharmaceutically acceptable salt or solvate thereof. The preparation comprises the process steps as described above and further comprises formulating the obtained telaprevir according to Formula 1or a pharmaceutically acceptable salt or solvate thereof (the aforementioned compounds may also be referred to as active pharmaceutically compounds, API) into a pharmaceutical composition or pharmaceutical dosage form. The step of formulating the API into a dosage form may be carried out by applying techniques known in the art. For example, the API can be formulated into tablets by using direct compression, dry or wet granulation processes, spray-coating processes or the like. The API may be formulated as an acid solution or as a solid as described in WO 2007/022459 A2 (paragraphs [0063]-[0064]).

A further aspect refers to a compound according to Formula 5, obtainable or obtained by carrying out steps (i) to (ii) of the process as described above. The compound according to Formula 5 has a high purity, preferably has an amount of epimeric impurity at the tert-leucine position of less than 1.0%, preferably less than 0.5%. The smallest amount of epimeric impurity may for example be 0.1%. The epimeric impurities can be determined by HPLC-MS or NMR as described above.

A further aspect refers to a compound according to Formula 7, obtainable or obtained by carrying out steps (i) to (iii) of the process as described herein. The compound according to Formula 7 has a high purity, preferably has an amount of epimeric impurity at the tert-leucine position of less than 1.0%, preferably less than 0.5%. The smallest amount of epimeric impurity may for example be 0.05%. The epimeric impurities can be determined by HPLC-MS or NMR as described above.

A further aspect refers to a compound according to Formula 6, obtainable or obtained by carrying out steps (i) to (iv) of the process as described above. The compound according to Formula 6 have a high purity, preferably have an amount of epimeric impurity at the tert-leucine position of below 1.0%, preferably below 0.5%. The smallest amount of epimeric impurity may for example be 0.05%. The epimeric impurities can be determined by HPLC or NMR as described above.

A further aspect relates to telaprevir according to Formula 1, a pharmaceutically acceptable salt or solvate thereof, obtainable or obtained by the process described herein.

Additionally preferred, telaprevir according to Formula 1 or pharmaceutically acceptable salt or solvate thereof, which is prepared by using the process described herein, contains a diastereomeric impurity at the tert-leucine position (cf. FIG. 2) of less than 0.15% and/or has a detectable amount of copper of above 0 ppm, such as 0.01 ppm or 0.05 ppm, and less than 1 ppm when using ICP-OES, i.e. above 0 to less than 1 ppm, 0.01 to less than 1 ppm or 0.05 to less than 1 ppm, wherein ICP-OES is described in the examples below.

### EXAMPLES

The following examples describe the present invention in detail, but are not to be construed to be in any way limiting for the present invention. In the examples below, the following abbreviations have the following meanings. Any abbreviations not defined have their generally accepted meaning. Unless otherwise stated, all temperatures are in degrees Celsius (° C.).

DMF: dimethylformamide;

EtOAc: ethyl acetate;

DCM: dichloromethane;

T3P: 2,4,6-Tripropyl-1,3,5,2,4,6-trioxatriphosphorinane-2,4,6-trioxide;

HOBt: 1-hydroxy-benzotriazole;

EDC: 1-Ethyl-3-(3-dimethylaminopropyl)carbodiimide hydrochloride;

PS-supported: Polystyrene-supported;

TEMPO: (2,2,6,6-Tetramethylpiperidin-1-yl)-oxyl;

Eq.: equivalents.

### Example 1

### Synthesis of Compounds According to Formula 5

### Example 1a

### (1S,3aR,6aS)-tert-butyl 2-((S)-2-((S)-2-cyclohexyl-2-(pyrazine-2-carboxamido)acetamido)-3,3 dimethylbutanoyfloctahydrocyclopenta[c]pyrrole-1-carboxylate (5a)

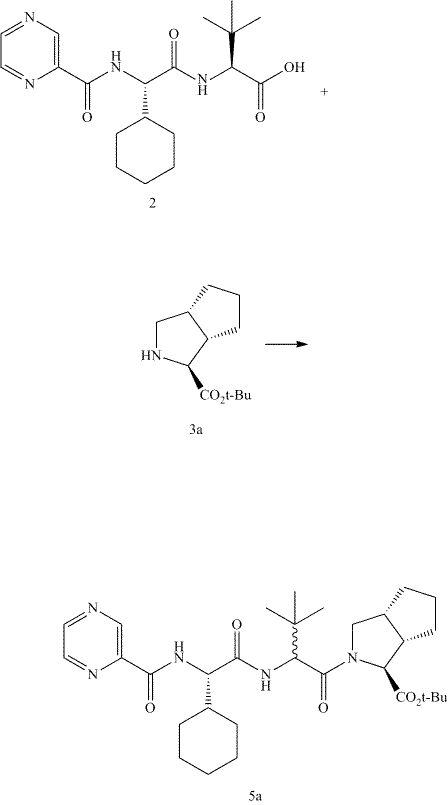 [SMILES: CC(C)(C)[C@H](NC(=O)[C@@H](NC(=O)c1cnccn1)C1CCCCC1)C(=O)O ; CC(C)(C)OC(=O)[C@H]1NC[C@@H]2CCC[C@@H]21 ; CC(C)(C)OC(=O)[C@@H]1[C@H]2CCC[C@H]2CN1C(=O)C(NC(=O)[C@@H](NC(=O)c1cnccn1)C1CCCCC1)C(C)(C)C]

A round bottom flask is charged with 40 mg of 2 (0.11 mmol, 1 eq.) and 1 mL of EtOAc is added. Then, 54 mg diisopropylethylamine (72 μl, 0.43 mmol, 4 eq) and 23 mg of 3a (0.11 mmol, 1 eq.) are added. After stirring for 5 min at 0° C., 78 μl of T3P (50% in EtOAc, 0.13 mmol, 1.2 eq.) are added and the reaction mixture is stirred for 3 h at 0° C., and for further 22 h at room temperature.

The reaction is then diluted with EtOAc, acidified to pH 2 using 2M HCl. After separation of the aqueous layer, the organic layer is washed with brine, dried over Na₂SO₄, filtered and concentrated in vacuo. Purification by flash chromatography yielded 5a (27 mg, 43% yield) as a 1:1.14 mixture of diastereomers. (d.r.=1:1.14).

### Example 1b

### (1S,3aR,6aS)-tert-butyl 2-((S)-2-((S)-2-cyclohexyl-2-(pyrazine-2-carboxamido)acetamido)-3,3 dimethylbutanoyfloctahydrocyclopenta[c]pyrrole-1-carboxylate (5b)

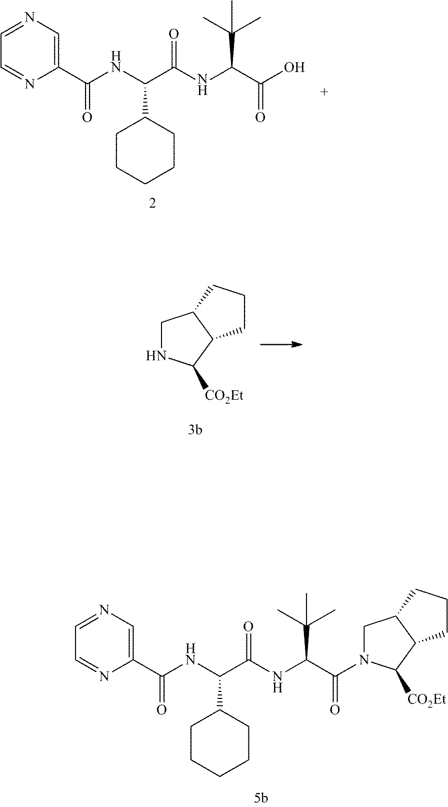 [SMILES: CC(C)(C)[C@H](NC(=O)[C@@H](NC(=O)c1cnccn1)C1CCCCC1)C(=O)O ; CCOC(=O)[C@H]1NC[C@@H]2CCC[C@@H]21 ; CCOC(=O)[C@@H]1[C@H]2CCC[C@H]2CN1C(=O)[C@@H](NC(=O)[C@@H](NC(=O)c1cnccn1)C1CCCCC1)C(C)(C)C]

A round bottom flask is charged with 41 mg of 2 (0.11 mmol, 1 eq.) and 1 mL of DCM is added. Then, 29 mg of 3b (0.16 mmol, 1.5 eq.) are added. After stirring for 5 min 190 μl of T3P (50% in EtOAc, 0.32 mmol, 3 eq.) are added and the reaction mixture is stirred for 21 h at room temperature.

The reaction is then diluted with DCM and quenched with water. The aqueous layer is separated and re-extracted with DCM. The combined organic layers are washed with brine, dried over Na₂SO₄, filtered and concentrated in vacuo. Purification by flash chromatography yielded 5b (26 mg, 43% yield). (d.r.=7.5:1).

### Example 1c

### (1S,3aR,6aS)-tert-butyl 2-((S)-2-((S)-2-cyclohexyl-2-(pyrazine-2-carboxamido)acetamido)-3,3 dimethylbutanoyl)octahydrocyclopenta[c]pyrrole-1-carboxylate (5b)

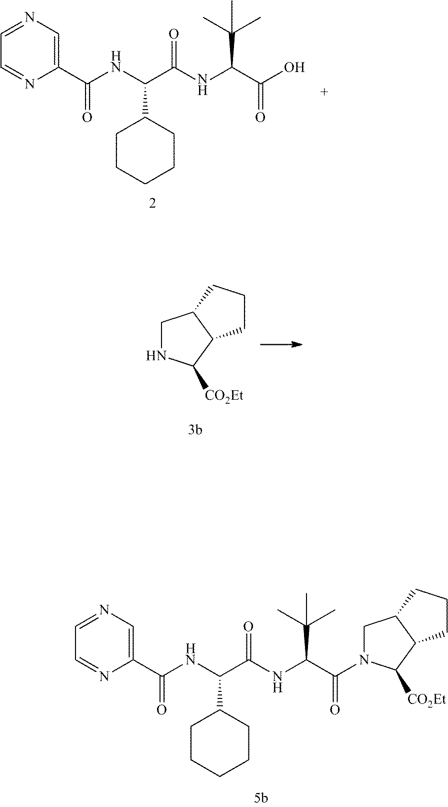 [SMILES: CC(C)(C)[C@H](NC(=O)[C@@H](NC(=O)c1cnccn1)C1CCCCC1)C(=O)O ; CCOC(=O)[C@H]1NC[C@@H]2CCC[C@@H]21 ; CCOC(=O)[C@@H]1[C@H]2CCC[C@H]2CN1C(=O)[C@@H](NC(=O)[C@@H](NC(=O)c1cnccn1)C1CCCCC1)C(C)(C)C]

A round bottom flask is charged with 1 g of 2 (2.66 mmol, 1 eq.) and 20 mL of DCM is added. Then, 0.73 g of 3b (3.98 mmol, 1.5 eq.) are added. After stirring for 5 min 4.75 mL of T3P (50% in EtOAc, 7.98 mmol, 3 eq.) are added and the reaction mixture is stirred for 21 h at room temperature.

The reaction is then quenched with water. The aqueous layer is separated and re-extracted with DCM. The combined organic layers are washed with sat. NaHCO₃-solution, brine and then dried over Na₂SO₄, filtered and concentrated in vacuo. Purification by flash chromatography yielded 5b (0.70 g, 49% yield). (d.r.=5.6:1).

### Example 1d

### (1S,3aR,6aS)-tert-butyl 2-((S)-2-((S)-2-cyclohexyl-2-(pyrazine-2-carboxamido)acetamido)-3,3 dimethylbutanoyl)octahydrocyclopenta[c]pyrrole-1-carboxylate (5b)

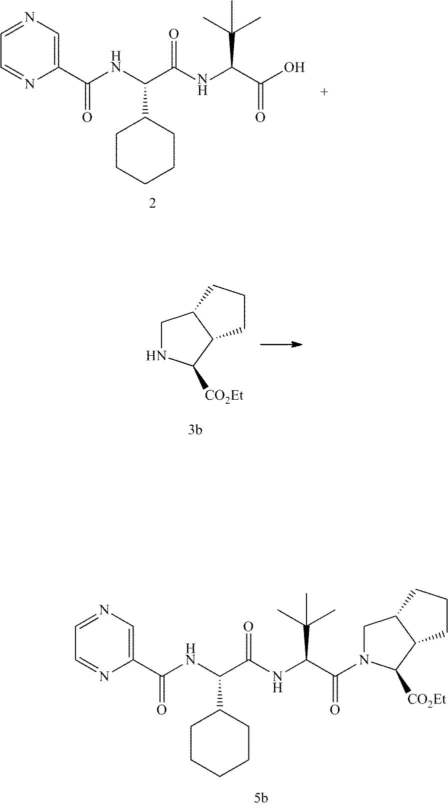 [SMILES: CC(C)(C)[C@H](NC(=O)[C@@H](NC(=O)c1cnccn1)C1CCCCC1)C(=O)O ; CCOC(=O)[C@H]1NC[C@@H]2CCC[C@@H]21 ; CCOC(=O)[C@@H]1[C@H]2CCC[C@H]2CN1C(=O)[C@@H](NC(=O)[C@@H](NC(=O)c1cnccn1)C1CCCCC1)C(C)(C)C]

A round bottom flask is charged with 1.0 g of 2 (2.66 mmol, 1 eq.) and 0.58 g of 3b (3.19 mmol, 1.2 eq), then 8 mL of DMF is added and the mixture cooled to 0° C. using an ice-bath. In a second flask, 0.36 g CuCl₂ (2.66 mmol, 1 eq.) are dispersed in 5 mL DMF, cooled to 0° C. and the previously prepared solution is added to it. Now, 0.36 g HOBt (2.66 mmol, 1 eq.) and 2.0 g EDC.HCl (10.43 mmol, 4 eq.) are added and the mixture is then stirred at r.t. for 16 h. The reaction is then quenched with 10 mL 10% NH₃-solution and then extracted 4 times with a total of 60 mL of EtOAc. The combined organic layers are then washed 3 times with dilute hydrochloric acid, once with sat. NaHCO₃-solution and brine, then dried over Na₂SO₄, filtered and concentrated in vacuo. Purification by flash chromatography yielded 5b (0.78 g, 54% yield). (d.r.=53:1).

### Example 1e

### (1S,3aR,6aS)-tert-butyl 2-((S)-2-((S)-2-cyclohexyl-2-(pyrazine-2-carboxamido)acetamido)-3,3 dimethylbutanoyl)octahydrocyclopenta[c]pyrrole-1-carboxylate (5b)

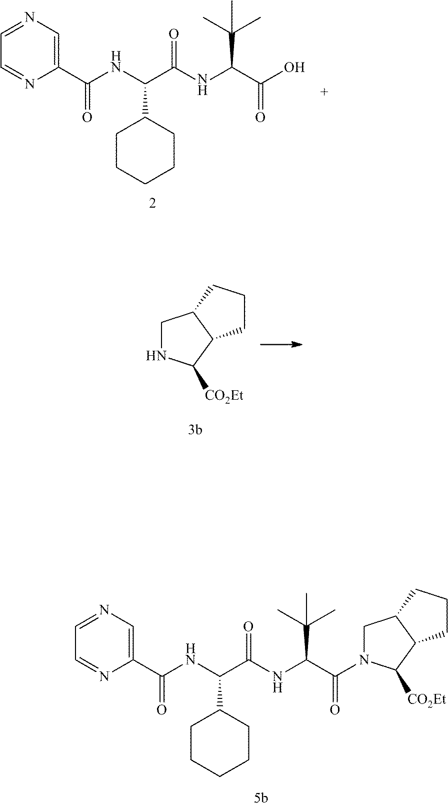 [SMILES: CC(C)(C)[C@H](NC(=O)[C@@H](NC(=O)c1cnccn1)C1CCCCC1)C(=O)O ; CCOC(=O)[C@H]1NC[C@@H]2CCC[C@@H]21 ; CCOC(=O)[C@@H]1[C@H]2CCC[C@H]2CN1C(=O)[C@@H](NC(=O)[C@@H](NC(=O)c1cnccn1)C1CCCCC1)C(C)(C)C]

A round bottom flask is charged with 1.25 g PS-supported HOBt (1.07 mmol/mg) and 0.30 g of 3b (1.65 mmol, 1.2 eq) and 0.36 g CuCl₂ (2.66 mmol, 1 eq.). Then 15 mL of DMF are added and the mixture cooled to 0° C. using an ice-bath, while mixing with a mechanical stirrer. In a second flask, 0.5 g of 2 (1.3 mmol, 1 eq.) and 1.0 g EDC.HCl (5.21 mmol, 4 eq.) are dispersed in 12 mL DMF, cooled to 0° C. and added to the previously prepared solution. The mixture is then stirred at r.t. for 22 h.

The reaction is then filtered and the filter washed with 15 mL DMF. 50 mL EtOAc are added to the filtrate, followed by 35 mL 5% NH₃-solution. The aqueous layer is then separated and extracted 3 times with a total of 45 mL of EtOAc. The combined organic layers are then washed once with 10% NH₃-solution, dilute hydrochloric acid, sat. NaHCO₃-solution and brine, then dried over Na₂SO₄, filtered and concentrated in vacuo. Purification by flash chromatography yielded 5b (0.43 g, 61% yield). (d.r.=18:1).

### Example 1f

### (1S,3aR,6aS)-tert-butyl 2-((S)-2-((S)-2-cyclohexyl-2-(pyrazine-2-carboxamido)acetamido)-3,3 dimethylbutanoyl)octahydrocyclopenta[c]pyrrole-1-carboxylate (5b)

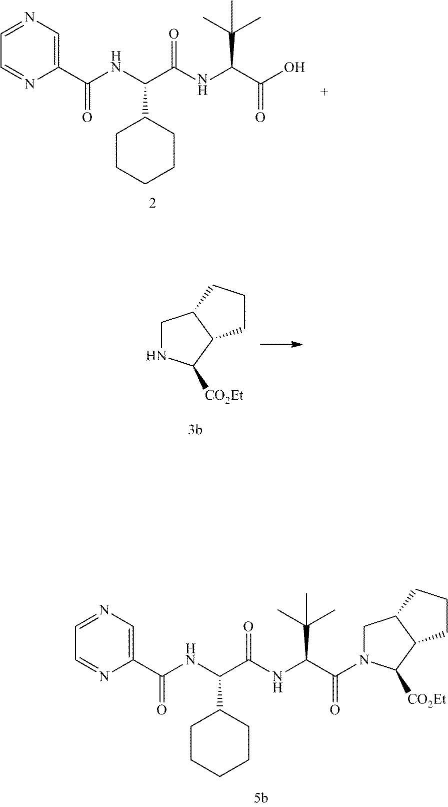 [SMILES: CC(C)(C)[C@H](NC(=O)[C@@H](NC(=O)c1cnccn1)C1CCCCC1)C(=O)O ; CCOC(=O)[C@H]1NC[C@@H]2CCC[C@@H]21 ; CCOC(=O)[C@@H]1[C@H]2CCC[C@H]2CN1C(=O)[C@@H](NC(=O)[C@@H](NC(=O)c1cnccn1)C1CCCCC1)C(C)(C)C]

A round bottom flask is charged with 2.0 g of 2 (5.3 mmol, 1 eq.) and 1.17 g of 3b (6.4 mmol, 1.2 eq), then 10 mL of DMF is added and the mixture cooled to 0° C. using an ice-bath, then 0.72 g CuCl₂ (5.3 mmol, 1 eq.) are added. In a second flask 0.72 g HOBt (5.3 mmol, 1 eq.) and 1.34 g DIC (10.6 mmol, 2 eq.) are dissolved in 5 mL DMF, cooled to 0° C. and added to the previously prepared solution. The mixture is then stirred at r.t. for 5 h.

The reaction is then quenched with 30 mL 2% NH₃-solution and then extracted 3 times with a total of 60 mL of EtOAc. The combined organic layers are then washed 3 times with a total of 60 mL of dilute hydrochloric acid, once with 20 mL sat. NaHCO₃-solution and 20 mL of brine, then dried over Na₂SO₄, filtered and concentrated in vacuo. Purification by flash chromatography yielded 5b (2.59 g, 90% yield). (d.r.=340:1)

### Example 1g

### Use of HOAT as Anti-Isomerisation Reagent

To 4.4 ml of a 0.6M HOAT solution in DMF (1.1 eq, 2.63 mmol) were added 0.6 ml DMF. Afterwards 1 g of 2 (90% content, 1 eq, 2.39 mmol), 567 mg of 3b (1.3 eq, 3.11 mmol) and 391 mg DIC (1.3 eq, 3.11 mmol) were added at room temperature. The reaction was stirred at room temperature for 23 h. After 19 h 86% conversion to 5b, with a d.r. 3.9/1 was observed. After 23 h, with 87% conversion to 5b, and a d.r. 4.1/1 the conversion had stalled and the product was not isolated.

### Example 1h

### Use of CuCl₂ with In Situ Generation of AOC-Et from its HCl Salt

353 mg water free CuCl₂ (1.1 eq, 2.63 mmol) was dissolved in 5 ml DMF. To the solution 1 g 2 (90% content, 1 eq, 2.39 mmol), 683 mg 3b. HCl (1.3 eq, 3.11 mmol), 315 mg NMM (1.3 eq, 3.11 mmol) and 391 mg DIC (1.3 eq, 3.11 mmol) were added at room temperature. The reaction mixture was stirred at room temperature. After two hours 2.6 area % 2 was detected and yield was 96.5% (calculated via internal standard). After 5 h less than 0.5 area % 2 was detected and yield was 98.1%. d.r. at both IPCs was 108/1.

### Example 1i

### HOAT Without CuCl₂ in DMF

To a solution of 0.5 g 2 (90%, 1 eq, 1.19 mmol) and 179 mg HOAT (1.1 eq, 1.32 mmol) in 2.5 ml DMF 284 mg 3b (1.3 eq, 1.55 mmol) was added. Afterwards 241 μl DIC (1.3 eq, 1.55 mmol) was added. Reaction was stirred at room temperature. After 2.5 h 91% conversion and DR of 4.3/1 was observed. After 5 h complete conversion Was observed and DR of 4.1/1. No work was performed.

### Example 1j

### HOAT Without CuCl₂ in THF/MED

To a suspension of 0.5 g 2 (90, 1 eq. 1.19 mmol) and 179 mg HOAT (1.1 eq, 1.32 mmol) in 2.5 ml of a 1/1 mixture of THF/MED (methylene chloride) 284 mg 3b (1.3 eq, 1.55 mmol) was added. Afterwards, 241 μl DIC (1.3 eq, 1.55 mmol) was added. Reaction was stirred at room temperature. After2.5 h 7.5% conversion and DR of 6.7/1 was observed. After 5 h 80 conversion was observed and DR of 6.2/1. After 19 h 86% conversion and DR of 6.0/1 was found. No work was performed.

### Example 1k

### HOAT with CuCl₂ in DMF

177 mg CuCl₂ (1.1 eq, 1.31 mmol) was dissolved in 2.5 ml DMF. To the solution 0.5 g 2 (90%, 1 eq, 1.19 mmol), 179 mg HOAT (1.1 eq, 1.32 mmol), 284 mg 3b (1.3 eq, 1.55 mmol) and 241 μl DIC (1.3 eq, 1.55 mmol) was added. The reaction was stirred at room temperature for 13 h. 95% conversion and DR of 48/1 was observed. No work up was performed.

### Example 1l

### Substochiometric Amounts of CuCl₂ in DMF Without HOAT

177 mg CuCl₂ (0.55 eq, 1.31 mmol) was dissolved in 5 ml DMF. To the solution 1.0 g 2 (90%, 1 eq, 2.39 mmol), 683 mg 3b. HCl (1.3 eq, 3.11 mmol), 340 μl NMM (1.3 eq, 3.11 mmol) and 481 μl DIC (1.3 eq, 3.11 mmol) was added. The reaction was stirred at room temperature for 4.5 h, complete conversion and DR of 76/1 was observed. No separated work up was performed.

### Example 2a

### Synthesis of the Compound According to Formula 7

### (1S,3aR,6aS)-2-((S)-2-((S)-2-cyclohexyl-2-(pyrazine-2-carboxamido)acetamido)-3,3-dimethylbutanoyl)octahydrocyclopenta[c]pyrrole-1-carboxylic acid (7)

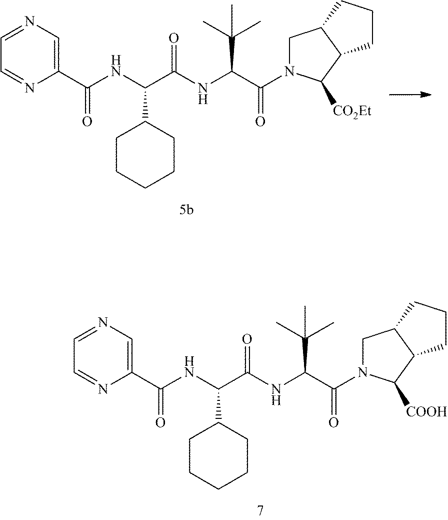 [SMILES: CCOC(=O)[C@@H]1[C@H]2CCC[C@H]2CN1C(=O)[C@@H](NC(=O)[C@@H](NC(=O)c1cnccn1)C1CCCCC1)C(C)(C)C ; CC(C)(C)[C@H](NC(=O)[C@@H](NC(=O)c1cnccn1)C1CCCCC1)C(=O)N1C[C@@H]2CCC[C@@H]2[C@H]1C(=O)O]

A round-bottom flask was charged with 6 g of 5b (11.08 mmol, 1 eq.) and 85 mL of THF and 26 mL of H₂O was added. Then 2.20 g LiOH.H₂O (52.43 mmol, 4.7 eq.) were added and the mixture was stirred at r.t. for 18 h.

Then 50 mL EtOAc and 50 mL H₂O were added, and the aqueous layer separated. The organic layer was washed once more with 40 mL H₂O. To the combined aqueous layers 50 mL of EtOAc were added, and by slow addition of 2M HCl the pH was adjusted to 1.89. After separation of the aqueous layer, it was extracted once more with 50 mL EtOAc, and the combined organic layers washed with brine, then dried over Na₂SO₄, filtered and concentrated in vacuo. Purification by flash chromatography yielded 7 (4.83 g, 85% yield).

### Example 2b

### Synthesis of the Compound According to Formula 7

### (1S,3aR,6aS)-2-((S)-2-((S)-2-cyclohexyl-2-(pyrazine-2-carboxamido)acetamido)-3,3-dimethylbutanoyl)octahydrocyclopenta[c]pyrrole-1-carboxylic acid (7)

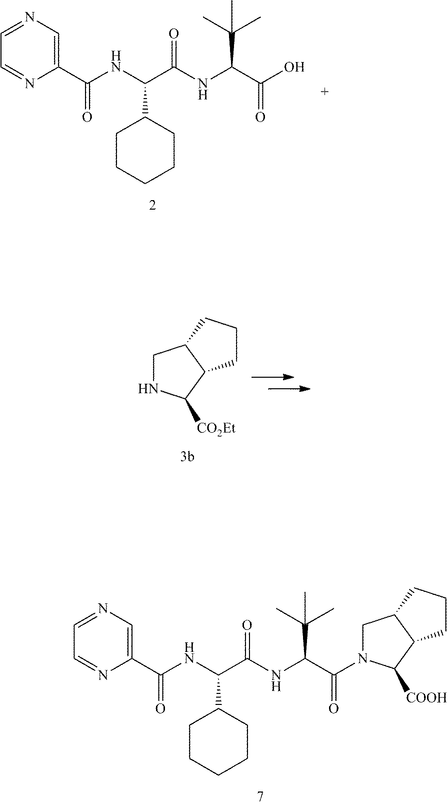 [SMILES: CC(C)(C)[C@H](NC(=O)[C@@H](NC(=O)c1cnccn1)C1CCCCC1)C(=O)O ; CCOC(=O)[C@H]1NC[C@@H]2CCC[C@@H]21 ; CC(C)(C)[C@H](NC(=O)[C@@H](NC(=O)c1cnccn1)C1CCCCC1)C(=O)N1C[C@@H]2CCC[C@@H]2[C@H]1C(=O)O]

A round bottom flask is charged with 2.0 g of 2 (5.3 mmol, 1 eq.) and 1.17 g of 3b (6.4 mmol, 1.2 eq), then 10 mL of DMF is added and the mixture cooled to 0° C. using an ice-bath, then 0.72 g CuCl₂ (5.3 mmol, 1 eq.) are added. In a second flask 0.72 g HOBt (5.3 mmol, 1 eq.) and 1.34 g DIC (10.6 mmol, 2 eq.) are dissolved in 3 mL DMF, cooled to 0° C. and added to the previously prepared solution. The mixture is then stirred at r.t. for 5 h.

The reaction is then quenched with 30 mL 2% NH₃-solution and then extracted 3 times with a total of 70 mL of EtOAc. The combined organic layers are then washed with 15 mL 2% NH₃-solution, 1 time with 20 mL 1M HCl, 3 times with a total of 60 mL of dilute hydrochloric acid, once with 20 mL sat. NaHCO₃-solution and 20 mL of brine, then dried over Na₂SO₄, filtered and concentrated in vacuo. The residue (compound 5b—2.59 g, 4.78 mmol, 1 eq.) was dissolved in 27 mL of a 1:1 mixture THF/H₂O. Then 0.48 g NaOH (11.95 mmol, 2.5 eq.) were added and the mixture was stirred at r.t. for 18 h.

Then 20 mL EtOAc and 10 mL H₂O were added, and the aqueous layer separated. The organic layer was washed once more with 20 mL H₂O. To the combined aqueous layers 20 mL of EtOAc were added, and by slow addition of 2M HCl the pH was adjusted to 1.27. After separation of the aqueous layer, it was extracted once more with 20 mL EtOAc, and the combined organic layers washed with brine, then dried over Na₂SO₄, filtered and concentrated in vacuo to give 7 (2.82 g, 93% yield). Trace metal analysis using ICP-OES showed residual copper<1 ppm, wherein the following method was used:

Digestion: about 250 mg of sample material was digested under pressure with a mixture of HNO₃+HCl in a closed quartz container which can be heated by microwave radiation.

Determination of Cu:

Measurement was performed with ICP-OES at 324,754 nm, Axialplasm, simultaneous background correction; calibration with external standards, certified elemental standard of Merck, Device: Fabr. Thermo Electron, Type: IRIS Intrepid XSP II, Duo.

### Example 2c

### Synthesis of the Compound According to Formula 7

### (1S,3aR,6aS)-2-((S)-2-((S)-2-cyclohexyl-2-(pyrazine-2-carboxamido)acetamido)-3,3-dimethylbutanoyl)octahydrocyclopenta[c]pyrrole-1-carboxylic acid (7)

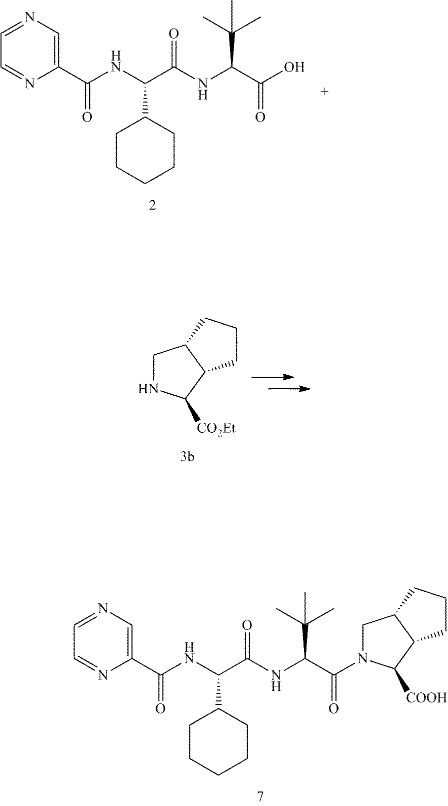 [SMILES: CC(C)(C)[C@H](NC(=O)[C@@H](NC(=O)c1cnccn1)C1CCCCC1)C(=O)O ; CCOC(=O)[C@H]1NC[C@@H]2CCC[C@@H]21 ; CC(C)(C)[C@H](NC(=O)[C@@H](NC(=O)c1cnccn1)C1CCCCC1)C(=O)N1C[C@@H]2CCC[C@@H]2[C@H]1C(=O)O]

7.07 g water free CuCl₂ (1.1 eq, 52.6 mmol) was dissolved in 100 ml DMF. To the solution 20 g of 2 (90% content, 1 eq, 47.82 mmol), 10.51 g of 3b (d.r.=9:1) (1.2 eq, 57.38 mmol), 6.3 ml NMM (1.2 eq, 57.38 mmol) and 9.6 ml DIC (1.3 eq, 62.16 mmol) were added at 0° C. the reaction mixture was warmed to 40° C. in 1.5 h and stirred at that temperature until complete conversion was observed. To the reaction mixture isopropyl acetate was added followed by the addition of 10% HCl. The organic phase was separated and washed with 5% ammonia and 2% NaCl. The organic solvent was removed to dryness and 26.95 g was isolated as a diasteromeric mixture of 9:1 detected via NMR.

6 g of this material was dissolved in 15 ml ethanol and 15 ml water. To the mixture 1.15 g sodium hydroxide was added. The reaction mixture was stirred at room temperature until no further conversion was observed. Ethanol was removed via distillation and water was added. The basic aqueous phase was washed with isopropyl acetate, the organic phase was re-extracted with water. To the combined aqueous phase Isopropyl acetate was added an pH was adjusted to 1.5 via addition of 10% HCl. The organic phase was separated and solvent was removed to dryness to yield 5.29 g of compound 7 as a single diastereomer according to NMR analysis.

### Example 2d

### Use of CuCl₂ as Anti Isomerisation Reagent (Without an Triazol Reagent)

353 mg water free CuCl₂ (1.1 eq, 2.63 mmol) was dissolved in 5 ml DMF. To the solution 1 g 2 (90% content, 1 eq, 2.39 mmol), 567 mg 3b (1.3 eq, 3.11 mmol) and 391 mg DIC (1.3 eq, 3.11 mmol) were added at room temperature. The reaction mixture was stirred at room temperature for 19 h full conversion to 5b with d.r. 116/1 was observed.

To the reaction mixture 50 ml of ethyl acetate was added and the occurring precipitation was removed via filtration. The organic phase was washed with 5% ammonia and the aqueous phase was reextracted with 50 ml ethyl acetate. The combined organic phase was washed with 40 ml 2M HCl and 40 ml brine. After drying with sodium sulfate and filtration the organic solvent was removed via evaporation. The solid residue was dissolved in 20 ml methylene chloride and again the solvent was removed to dryness. After drying (rt, 40 mbar), 1.373 g of a slightly yellow amorphous solid (NMR content 81.6%, yield 86.4%).

### Example 3

### Synthesis of the Compound According to Formula 6

### (1S,3aR,6aS)-2-((S)-2-((S)-2-cyclohexyl-2-(pyrazine-2-carboxamido)acetamido)-3,3-dimethylbutanoyl)-N-((S)-1-(cyclopropylamino)-2-hydroxy-1-oxohexan-3-yl)octahydrocyclopenta[c]pyrrole-1-carboxamide (6)

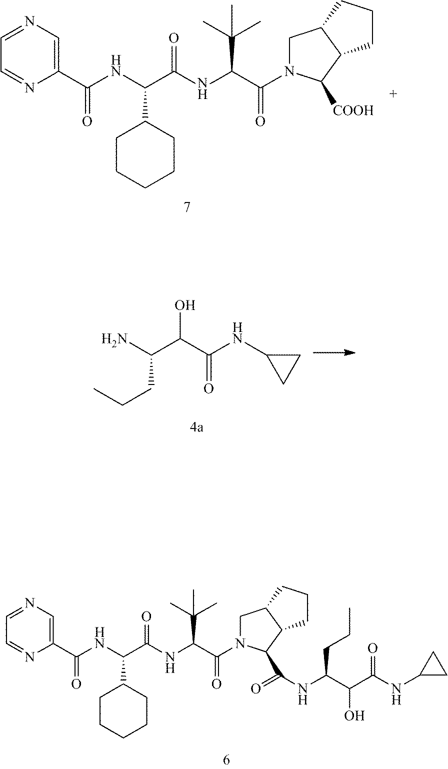 [SMILES: CC(C)(C)[C@H](NC(=O)[C@@H](NC(=O)c1cnccn1)C1CCCCC1)C(=O)N1C[C@@H]2CCC[C@@H]2[C@H]1C(=O)O ; CCC[C@H](N)C(O)C(=O)NC1CC1 ; CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(O)C(=O)NC1CC1]

A round-bottom flask was charged with 11.87 g of 7 (23.11 mmol, 1 eq.), 5.32 g of EDC.HCl (27.73 mmol, 1.2 eq.), 3.74 g of HOBt (27.73 mmol, 1.2 eq.) and 80 mL DCM were added. The mixture was cooled with an ice-bath and a suspension of 5.66 g of 4 (25.42 mmol, 1.1 eq.) in 50 mL of DCM containing 2.75 g NEt₃(27.73 mmol, 3.88 mL, 1.2 eq.) was added. This mixture was then stirred at r.t. for 6 h when conversion was complete.

The reaction was quenched by addition of 50 mL H₂O, followed by dropwise addition of 6M HCl to adjust the pH to 1.45. The aqueous layer was separated and extracted once with 50 mL DCM. The combined organic layers were washed with 50 mL sat. NaHCO₃ solution and 50 mL brine, dried over Na₂SO₄, filtered and concentrated in vacuo. Purification by flash chromatography yielded 6 (14.89 g, 94% yield).

### Example 4

### Synthesis of the Compound According to Formula 1

### (1S,3aR,6aS)-2-((S)-2-((S)-2-cyclohexyl-2-(pyrazine-2-carboxamido)acetamido)-3,3-dimethylbutanoyl)-N-((S)-1-(cyclopropylamino)-1,2-dioxohexan-3-yl)octahydrocyclopenta[c]pyrrole-1-carboxamide (Telaprevir) (1)

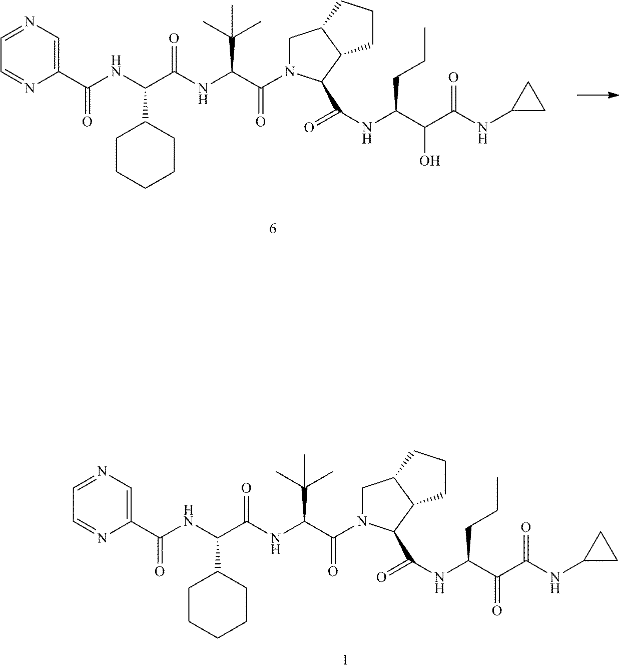 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(O)C(=O)NC1CC1 ; CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

A round-bottom flask was charged with 2.00 g of 6 (2.93 mmol, 1 eq.) and 20 mL of DCM and then cooled with an ice-bath. 200 μl of 15% KBr-solution and 800 μl of sat. NaHCO₃-solution were added, followed by 11 mg of TEMPO (0.07 mmol, 0.025 eq.) and 600 μl 10% NaOCl-solution. After stirring at r.t. for 18 h, another 1.2 mL of 10% NaOCl-solution were added—after another 2 h the reaction was complete.

The reaction mixture was then diluted with 10 mL of H₂O. After separation of the aqueous layer it was extracted with 10 mL of DCM. The combined organic layers were washed with 10 mL of 1% Na₂SO₃ and 10 mL of H₂O, dried over Na₂SO₄, filtered and concentrated in vacuo. The residue was then stirred in 40 mL Et₂O, filtered, washed with 10 mL of cold Et₂O and then dried in vacuo to give crystalline 1 (1.41 g, 71%).

### CITATION LITERATURE

WO 2007/022459 A2; Turner et al. (Chemical Communications 2010, 46(42), 7918); WO2010/126881; Y. Yip et al. Bioorg. Med. Chem. Lett., 2004, 14, 5007; Harbeson, S. L. et al. J. Med. Chem. 1994, 37, 2918-2929.

1. Process for the preparation of telaprevir according to Formula 1 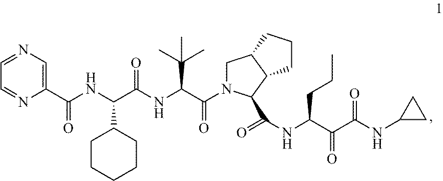 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]
or a pharmaceutically acceptable salt or solvate thereof, comprising the steps of:
i) providing a compound according to Formula 2 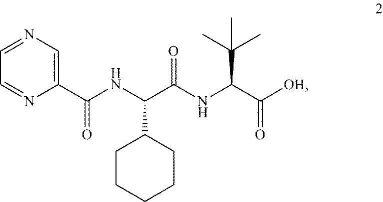 [SMILES: CC(C)(C)[C@H](NC(=O)[C@@H](NC(=O)c1cnccn1)C1CCCCC1)C(=O)O]
wherein the compound of Formula 2 has a diastereomeric purity of at least 70% based on the total amount of all isomers of Formula 2,
ii) bringing the compound according to Formula 2, into contact with a compound according to Formula 3 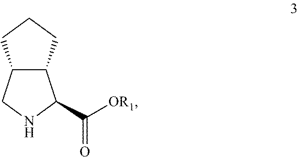
wherein R₁ is a protection group and the compound of Formula 3 has a stereochemical purity of at least 70% based on the total amount of all isomers of Formula 3, in the presence of one or more coupling agents and a lewis acid, thereby obtaining a compound according to Formula 5, 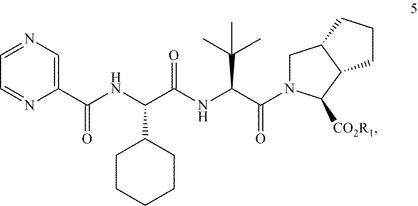
iii) deprotecting the compound according to Formula 5 by removing the R₁ protection group in order to obtain an acid according to Formula 7 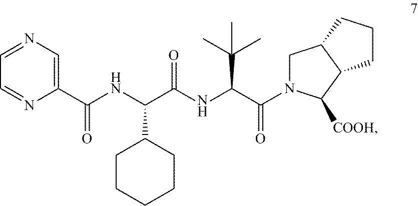 [SMILES: CC(C)(C)[C@H](NC(=O)[C@@H](NC(=O)c1cnccn1)C1CCCCC1)C(=O)N1C[C@@H]2CCC[C@@H]2[C@H]1C(=O)O]
iv) bringing the acid according to Formula 7 into contact with a compound according to Formula 4 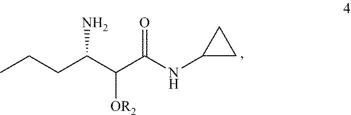
wherein R₂ is H, or optionally, a suitable protecting group in the presence of one or more coupling agents, thereby obtaining a compound according to Formula 6 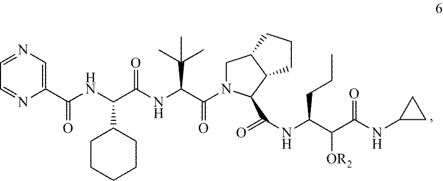
wherein R₂ is H, or optionally, a suitable protecting group followed by removal of the optionally present protecting group R₂, thereby obtaining a compound according to Formula 6 wherein R₂ is H; and
v) oxidizing the compound according to Formula 6, thereby obtaining telaprevir according to Formula 1, or a pharmaceutically acceptable salt or solvate thereof.

2. The process of claim 1, wherein step (iii) further comprises extracting a product into the aqueous layer using an aqueous base and/or wherein R₁ in the compounds according to Formula 3 and 5 is a saturated or unsaturated, cyclic, linear or branched, substituted or unsubstituted C₁₋₁₀ hydrocarbon compound.

3. The process of claim 1, wherein step (ii) further comprises the use of a solvent selected from the group consisting of ethylacetate, dichloromethane, N,N-dimethylacetamide, dimethyl sulfoxide, N-methylpyrrolidone, acetonitrile, methyl tert-butyl ether, methyltetrahydrofuran, toluene and dimethylformamide.

4. The process of claim 1, wherein the coupling agent used in step (ii) comprises a compound according to Formula 8 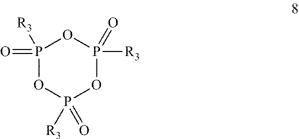
wherein R₃ is a saturated or unsaturated, cyclic, linear or branched, substituted or unsubstituted C₁₋₁₀ hydrocarbon compound; or a carbodiimide.

5. The process of claim 1, wherein the coupling agent used in step (ii) comprises at least one coupling agent selected from the group consisting of 2,4,6-tripropyl-1,3,5,2,4,6-trioxatriphosphorinane-2,4,6-trioxide, 2,4,6-triphenyl-1,3,5,2,4,6-trioxatriphosphorinane-2,4,6-trioxide, N,N′-diisopropylcarbodiimide, 1-ethyl-3-(3-dimethylaminopropyl)carbodiimide hydrochloride and N,N′-dicyclohexylcarbodiimide.

6. The process of claim 1, wherein a second coupling agent is used.

7. The process of claim 1, wherein N,N′-diisopropylcarbodiimide and 1-hydroxy-benzotriazole are used as coupling reagents, and dimethylformamide is used as solvent.

8. The process of claim 1, wherein the lewis acid in step (ii) comprises a copper salt.

9. The process of claim 1, wherein the oxidizing agent used in step (v) is selected from the group of hypervalent iodine oxidants, comprising the Dess-Martin periodinane (1,1,1-Tris(acetyloxy)-1,1-dihydro-1,2-benziodoxol-3-(1H)-one) or IBX (2-iodoxybenzoic acid), or sodium hypochlorite in the presence of 2,2,6,6-tetramethylpiperidinyloxy free radical (TEMPO).

10. The process of claim 1, wherein the telaprevir according to Formula 1, a pharmaceutically acceptable salt or solvate thereof is obtained in amorphous form, crystalline form or as cocrystals.

11. Process for the preparation of a compound according to Formula 5 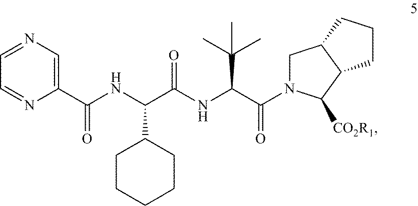
comprising the steps of:
i) providing a compound according to Formula 2 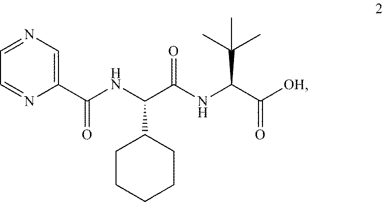 [SMILES: CC(C)(C)[C@H](NC(=O)[C@@H](NC(=O)c1cnccn1)C1CCCCC1)C(=O)O]
wherein the compound of Formula 2 has a diastereomeric purity of at least 70% based on the total amount of all isomers of Formula 2,
ii) bringing the compound according to Formula 2 into contact with a compound according to Formula 3 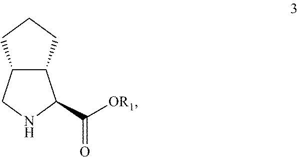
wherein R₁ is a protection group and the compound of Formula 3 has a stereochemical purity of at least 70% based on the total amount of all isomers of Formula 3, in the presence of one or more coupling agents and a lewis acid, thereby obtaining a compound according to Formula 5.

12. Process for the preparation of a pharmaceutical composition or pharmaceutical dosage form comprising telaprevir according to Formula 1 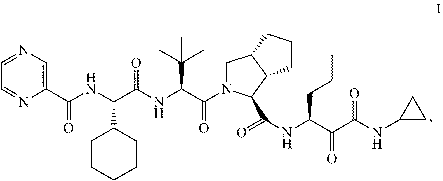 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]
or a pharmaceutically acceptable salt or solvate thereof, having a detectable amount of copper of above 0 ppm and less than 1 ppm when using ICP-OES, comprising the process steps as defined in claim 1 and further comprising formulating the obtained telaprevir according to Formula 1, or a pharmaceutically acceptable salt or solvate thereof into a pharmaceutical composition or pharmaceutical dosage form.

13. Crystalline compound according to Formula 7 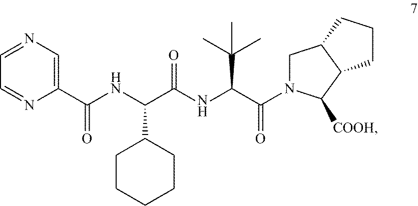 [SMILES: CC(C)(C)[C@H](NC(=O)[C@@H](NC(=O)c1cnccn1)C1CCCCC1)C(=O)N1C[C@@H]2CCC[C@@H]2[C@H]1C(=O)O]
obtainable or obtained by carrying out steps (i) to (iii) of the process as defined in claim 1.

14. (canceled)

15. The process of claim 6, wherein the second coupling agent is 1-hydroxy-benzotriazole or 1-hydroxy-7-aza-benzotriazole.

16. The process of claim 8, wherein the lewis acid in step (ii) is CuCl₂.

17. The process of claim 9, wherein the oxidizing agent used in step (v) is sodium hypochlorite in the presence of 2,2,6,6-tetramethylpiperidinyloxy free radical (TEMPO).

18. The process of claim 12, wherein the lewis acid in step (ii) is a copper salt.

19. The process of claim 12, wherein the lewis acid in step (ii) is CuCl₂.
